(19) United States
(12) Patent Application Publication
(10) Pub. No.: US 2010/0036289 A1
(43) Pub. Date: Feb. 11, 2010

(54) Apparatus and Methods for Assessment of Vestibulo-Ocular Reflexes

(75) Inventors: Keith D. White, Gainesville, FL (US); Tana Marie Bleser, Archer, FL (US); Erika Espinoza Williams, Port Richey, FL (US); John Bacom, High Springs, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: UNIVERSITY OF FLORIDA RESEARCH FOUNDATION, INC., Gainesville, FL (US)

(21) Appl. No.: 12/513,854

(22) PCT Filed: Jan. 15, 2008
(86) PCT No.: PCT/US2008/051049

§ 371 Date: Jul. 6, 2009

Related U.S. Application Data
(60) Provisional application No. 60/886,683, filed on Jan. 26, 2007.

(51) Int. Cl. A61B 5/11

(52) U.S. Cl. 600/595

(57) ABSTRACT

A rotary disk securely rotates a patient for the purpose of inducing vestibulo-ocular reflex eye movements. The apparatus provides a rotating platform with a positioning post to which a patient can be secured with a safety vest. A headrest on the positioning post permits a patient's head to be positioned at an angle conducive to inducing reflex eye movements. A camera or other recording device is used to view and record eye movements before, during and/or after a patient is subjected to a period of rotation.

### BACKGROUND OF INVENTION

The vestibular system is the primary sensory system that controls balance and spatial orientation in most mammals, including humans. Located within the inner ear, the vestibular system includes the semi-circular canals and the otolith organs. The vestibular system significantly influences the muscles of the eye and the muscles that control posture and balance of the body

Signals arising from the vestibular system influencing the eye-muscles help maintain stable vision despite head rotation through static (activated by head posture) and dynamic (activated by head motion) components. Known collectively as vestibulo-ocular reflexes (VOR), they are among the fastest reflexes in the body. In humans, the neural connection between the vestibular system and the eye muscles comprises just three neurons providing for delays on the order of 10 milliseconds between head movements and the adjustment of the eyes to that movement. The speed of this connection minimizes retinal image displacements caused by head movements. The “doll reflex”, downward rolling of the eyes in response to pitching the upright head backward, and ocular counter-rolling, torsional eye posture changes in response to tilting an ear toward the shoulder, are examples of static vestibulo-ocular reflexes. Dynamic vestibulo-ocular reflexes normally maintain eye fixation on an attended image during small movements of the head

Nystagmus is a manifestation of dynamic vestibulo-ocular reflexes during extensive head rotations such as full revolutions. Nystagmus is characterized by repetitive slow eye movements in one direction interleaved with quick eye movements in the opposite direction. The vestibular system, stimulated by head angular velocity, counter-rotates the eyes to their rotational limit within the orbit whereupon the eyes reset quickly to straight ahead, and this slow-quick pattern repeats creating nystagmus. The inability to induce nystagmus in a person can indicate an underlying physiological or pathological condition, as can spontaneously occurring nystagmus.

There are many different types of nystagmus caused by a variety of physiological or pathological factors. For example, the “Horizontal Gaze Nystagmus” test is often used as one indicator of an elevated blood alcohol level. Other pathologies that may be indicated by various abnormalities of nystagmus are head trauma, brain tumors, albinism, stroke, multiple sclerosis, certain dietary deficiencies or congenital disorders, and diseases such as Pelizaeus-Merzbacher disease. Of particular interest is the characterization of these eye movements in children, which can indicate congenital or acquired neurological or neuromuscular disease. If detected and treated early, the progression of disorders can sometimes be reversed or lessened. Because of its diagnostic power, the ability to characterize vestibulo-ocular reflexes can potentially provide a valuable and reliable test.

Current testing procedures often utilize devices that rotate or simulate other movement of the whole body. Often, these devices are manually operated by the observer. For example, the Southern California Post-Rotary Nystagmus Test is designed for use with children. It utilizes a large disk, like a “lazy susan” that is manually rotated while the patient being tested is seated thereon. The patient is usually asked to lean forward and support themselves on the edge of the disk to approximate the desired head angle to preferentially stimulate the horizontal semi-circular canal. Other devices, such as, for example the “Barany chair” also rotate, but are designed for adult test patients. If used with pediatric patients, they must usually be seated in an adult's lap in the chair.

In addition, current methods of observing or recording eye movements frequently utilize electrodes, goggles or other contact devices. Often, these devices are not comfortable to, or tolerated well by, pediatric patients with disabilities such as autism. For some children, devices that contact their face are especially objectionable.

The subject invention provides an apparatus and methods for eliciting eye movement responses, such as nystagmus, mediated through vestibulo-ocular reflexes by postural or rotational activation of the vestibular sensory system. The apparatus allows consistent, safe positioning of patients, providing a constant stimulus across subjects for systematic testing. Also provided are non-invasive apparatuses and methods for observing and recording a patient's eye postures and eye movements before, during, and after subjection to rotational activation.

### BRIEF SUMMARY

The methods and apparatus of the subject invention are used for eliciting eye movement responses mediated through vestibulo-ocular reflexes (VOR). Ocular counter-rolling, a static vestibulo-ocular reflex, results from cyclotorsional eye movements made in response to adopting a posture with the mid-sagittal plane of the head out of the gravitational vertical by tilting an ear toward the shoulder. Nystagmus eye movements are made in response to rotational activation of the vestibular sensory system. In one embodiment, the apparatus comprises a “rotary disk” upon which a subject can be positioned and rotated to stimulate the horizontal semicircular canals of the vestibular sensory system, which elicits a dynamic vestibulo-ocular reflex eye movement, such as, for example, nystagmus. Also provided is a means for viewing and/or recording eye posture during head tilt (ocular counter-rolling) and a means for viewing and/or recording eye movements of a patient during rotation (per rotary) and after rotation (post rotary).

To achieve consistency between tested patients, the stimulation provided by the rotary disk should be applied similarly to tested patients. For example, appropriate downward tilt of the head causes preferential stimulation of the horizontal semi-circular canals of the vestibular system during rotation, which elicits the dynamic vestibulo-ocular eye reflex known as horizontal nystagmus. Rightward or leftward tilt of the head causes postural stimulation of the otolith organs of the vestibular system, which elicits the static vestibulo-ocular eye reflex known as ocular counter-rolling. Therefore, it can be helpful to ensure that all patients tested are positioned with substantially the same body positions, e.g., head tilts, to ensure that counter-rolling and nystagmus are achieved consistently between tested patients. Thus, also provided by the subject invention is a central support and headrest with which a patient can be positioned, not only to achieve consistency in testing, but also to provide a safe way to secure patients on the rotary disk when it is in motion.

Further, the rate of rotation, or angular velocity, can also be a factor in eliciting vestibulo-ocular nystagmus responses in a patient. Thus, as with a patient's positioning, a consistent rotation speed, or angular velocity stimulus, can also ensure accurate and consistent test results between individuals. Therefore, one embodiment of the subject invention provides a controlled, motorized rotary disk for consistent clockwise or counter-clockwise rotation. In a further embodiment, the motorized rotary disk is remotely controlled. In a still further embodiment, the motorized rotary disk may be controlled by computer and/or software.

A further advantage of the subject invention is the ability to observe eye movements via a non-invasive apparatus. In one embodiment, the subject invention utilizes a digital still camera mounted to the rotary disk support and adjusted to view and record eye postures of a patient in response to upright, rightward, or leftward tilted head postures. In a second embodiment, the subject invention utilizes a video camera mounted to the rotary disk support and adjusted to view and record eye movements of a patient at any time during the testing routine. In this embodiment, the video camera rotates with the patient so that “per rotary”, as well as “post rotary”, eye movements can be observed and/or recorded.

### BRIEF DESCRIPTION OF DRAWINGS

In order that a more precise understanding of the above recited invention is obtained, a more particular description of the invention briefly described above will be rendered by reference to specific embodiments thereof that are illustrated in the appended drawings. Understanding that these drawings depict only typical embodiments of the invention and are not therefore to be considered as limiting in scope, the invention will be described and explained with additional specificity and detail through the use of the accompanying drawings in which:

FIG. 1 is a photograph of an embodiment of the rotary disk device of the subject invention. Shown are the rotary disk platform and base and the attached remote control.

FIG. 2 is a photograph of an embodiment of the rotary disk device of the subject invention. Shown are the rotary disk platform and base with the positioning post, head rest, mounted video camera and remote control.

FIG. 3 is a photograph of a patient seated on an embodiment of the rotary disk device of the subject invention. Shown is the subject wearing a padded safety harness or vest that secures, via, straps, the individual to the padded central support, while the head is comfortably secured to or rested on the headrest.

FIG. 4 is a photograph of the side view of the patient shown in FIG. 3 illustrating how the subject is secured to the central post, arms optionally around the post, and the head positioned and comfortably secured at the desired angle, such that the attached video camera is able to observe and record the subject's eye movements.

FIG. 5 is a photograph of a front view of the individual in FIG. 3. From this photograph, it call be seen that the video camera OF one embodiment of the subject invention is able to observe and/or record the eye movements of a patient, before, during, and/or after, rotation by the rotary disk.

FIGS. 6A, 6B, 6C, and 6D are a series of photographs that show how a patient, secured to an embodiment of the subject invention, can be rotated by the rotary disk and the subject's head secured such that the video camera, or similar device, is able to record eye movements.

FIGS. 7A, 7B, and 7C are a series of photographs that show selected involuntary positions of the eye during a horizontal nystagmus event.

### DETAILED DISCLOSURE

The subject invention in general provides embodiments of an apparatus for inducing and/or observing a vestibulo-ocular reflex (VOR). More specifically, the subject invention pertains to one or more embodiment(s) of a rotary disk apparatus capable of rotating a patient, usually for the purpose of inducing a nystagmus eye movement response, or of positioning a patient in a suitable posture for inducing an ocular counter-rolling response.

The rotary disk of the subject invention can be particularly useful to pediatric neurologists for assessing suspected vestibular dysfunction or suspected brain lesion involving the pathways that mediate vestibulo-ocular reflexes (vestibular nuclei, medial longitudinal fasciculus, oculometer, trochlear and abducens nuclei). In addition, evidence indicates that the vestibulo-ocular reflexes, for example, post-rotary nystagmus, may be altered in autistic individuals, and, thus, may be detectable with the devices and methods of the subject invention.

The subject device and methods can further be utilized by therapists and vestibular/balance clinicians. Also, a person with skill in the art will be able to recognize numerous other uses for the rotary disk, including uses on or for adults that would be applicable to the devices and methods of the subject invention. While the subject application describes a use of the rotary disk for inducing and/or observing vestibulo-ocular reflex events, other modifications apparent to a person with skill in the art and having benefit of the subject disclosure are contemplated to be within the scope of the present invention.

The term “patient” or “subject” as used herein, describes an animal, including mammals, to which the systems and methods, or appropriate modifications thereof of the present invention are applied. Mammalian species that can benefit from the disclosed systems and methods include, but are not limited to, apes, chimpanzees, orangutans, humans, monkeys; domesticated animals (e.g., pets) such as dogs, cats, guinea pigs, hamsters; veterinary uses for large animals such as cattle, horses, goats, sheep; and any wild animal for veterinary or tracking purposes.

The terms “physician” or “clinician” as used in the subject invention are merely for literary convenience. The terms should not be construed as limiting in any way. The devices, apparatuses, methods, techniques and/or procedures of the subject invention could be utilized by any person desiring or needing to do so and having the necessary skill and understanding of the invention.

Also, as used herein, and unless otherwise specifically stated, the terms “operable communication” and “operably connected” mean that the particular elements are connected in such a way that they cooperate to achieve their intended function or functions. The “connection” may be direct, or indirect, physical or remote.

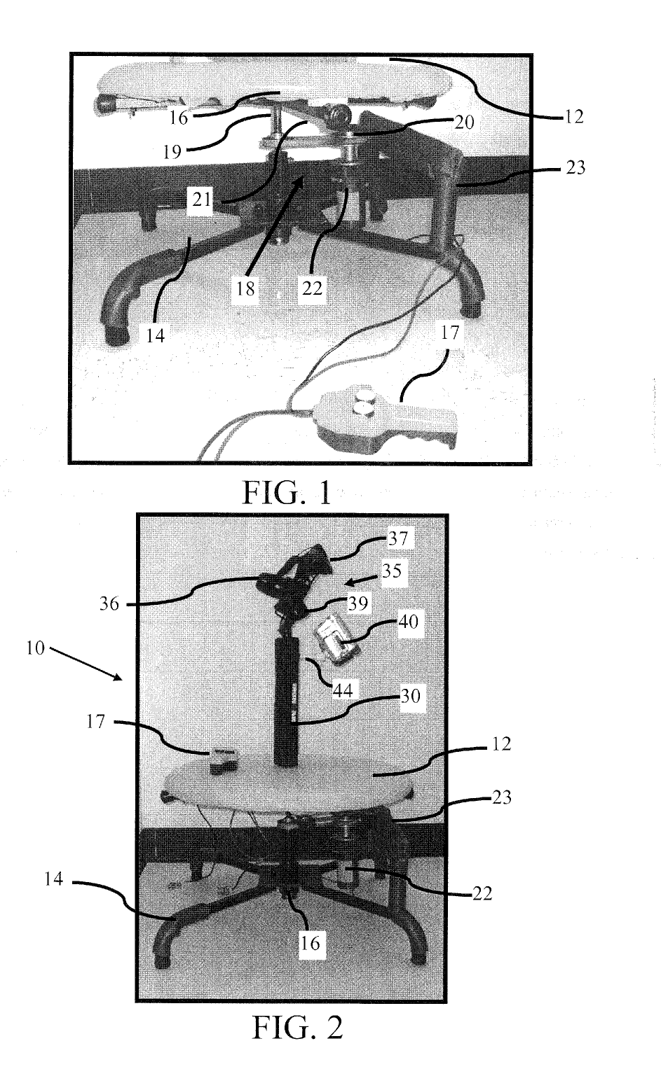

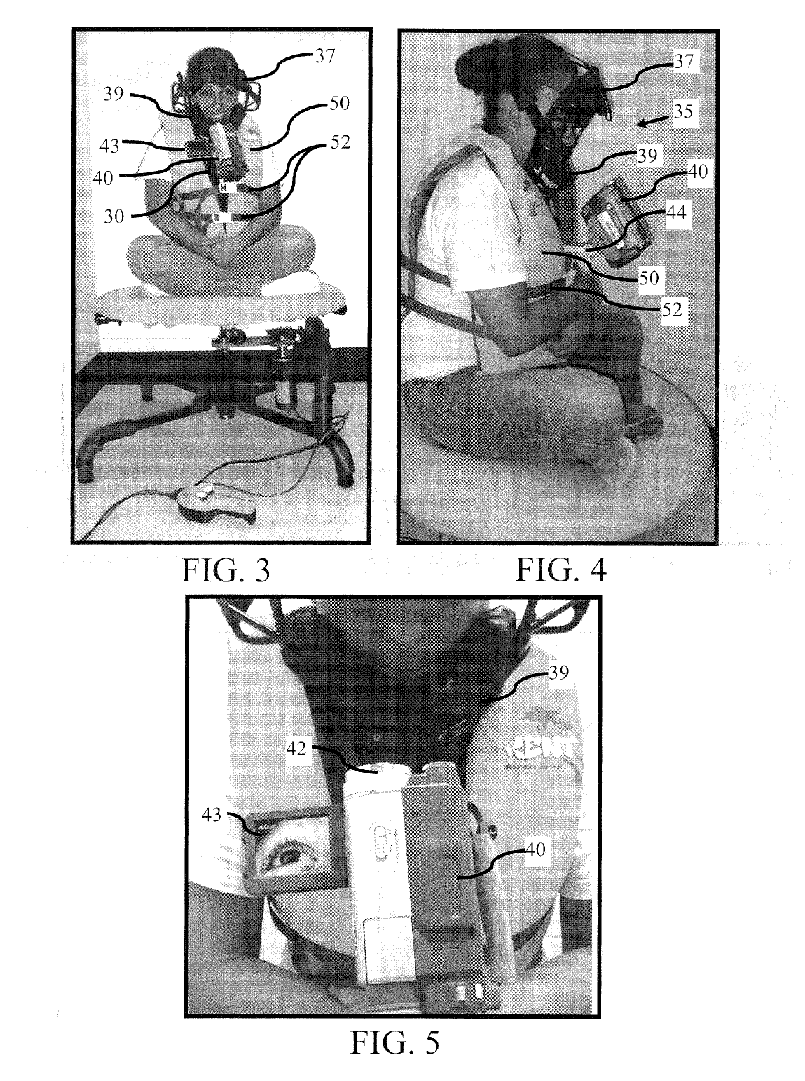

With reference to the attached figures, which show certain embodiments of the subject invention, it can be seen that the rotary disk 10 of the subject invention comprises a platform 12 rotatably attached to a base 14, for example as shown in FIG. 1. The platform 12 can comprise any of a variety of circumferential shapes, including, but not limited to, circular, oval, square, triangular, or ally other polygonal shape suitable for accommodating a patient positioned thereon. In a preferred embodiment, the platform 12 has a circular circumferential shape. The dimensions of the platform 12 are also such for accommodating a patient positioned thereon. In a preferred method, the patient is seated on the platform, with cross-legs, as shown in FIG. 3. However, it may be preferable in some circumstances for a patient to attain a different posture on the platform, such as kneeling or sitting on folded legs, sitting with legs straight out, etc. Thus, the size of the platform 12 should be adequate to accommodate various postures that may be required for different types of testing and postures.

In a preferred embodiment, a patient would sit cross-legged at or near the center of the platform 12. But, in alternative embodiments, a patient may be positioned lying face-up on the platform with their head centered on the platform, such that their body can be rotated with their head rotating at or near the center of the platform. To accommodate these postures, the diameter of the platform can be from approximately 1.5 ft to approximately 12 ft. In a more preferred embodiment, the diameter of the platform can be from approximately 2.5 ft. to about 3.0 ft. In a still further preferred embodiment, the platform 12 may comprise a variety of paddings, cushioning, or contouring to aid in the comfort, positioning and/or testing of a patient.

The platform 12 is rotatably attached to a base 14 that is capable of supporting the platform 12 and a patient positioned thereon. The base 14 can comprise any of a variety of configurations known to those with skill in the art. For example, the base 14 can comprise 3 or more legs, as shown, for example, in FIG. 1. Alternative embodiments can utilize additional legs, a barrel-style base, a disk-style base, etc. In a preferred embodiment, the style of the base 12 should be such to adequately and securely support the platform 12 and a patient positioned and being rotated thereon. Alternative embodiments, can further utilize various types of padding, cushioning, contouring, etc. for the safety and/or positioning of a patient. Further alternative embodiments, can include one or more steps or gradations to assist a patient in attaining the level of the platform 12 for positioning thereon. And, still further alternative embodiments, can utilize various auxiliary supports or fasteners, clamps, couplings, bolts, etc. to secure the base 14 to a floor or other structure. A person with skill in the art would be able to determine a wide variety of additional supports or means of securing the base 14 of the subject invention and are contemplated to be within the scope of the subject invention.

The platform 12 can be connected to the base 14 by a rotatable vertical axle 16. In a preferred embodiment, the vertical axle 16 is positioned at or near the center of the platform 12 and the base 14. This can provide stability and uniform rotation, or angular velocity, to the rotary disk 10. However, alternative configurations would be apparent to a person with skill in the art and such comparable alternatives should be considered within the scope of the subject invention. By way of a further, non-limiting example, the vertical axle 16 may be adjustable, permitting the height of the platform 12 to be changed relative to the base 14.

To induce nystagmus, as discussed above, a patient positioned on the platform 12 is usually rotated in a clockwise or counter-clockwise direction for a short period of time. Thus, in a further embodiment of the rotary disk 10 of the subject invention, the platform 12 can be rotated in a clockwise or counter-clockwise direction. However, to achieve consistency in testing, it is preferable that the rotation of the platform be consistent during a test and between individual tests. Any of a variety of apparatuses and/or methods, apparent to one with skill in the art, can be used to achieve a consistent rotation of the platform 12.

In a preferred embodiment, the rotation, or angular velocity, of the platform 12 is motor-driven. For example, one embodiment, shown in FIG. 1, utilizes a pulley system 18 comprising an axle pulley 19 operably connected to a motor pulley 20 by a pulley belt 21. The axle pulley 19 is located on the vertical axle 16 supporting the platform 12. The motor pulley 20 is operably connected to a motor 22. In a preferred embodiment, the motor pulley 20 is driven by an electric motor. In a more preferred embodiment, the motor pulley 20 is driven by a 12V DC motor.

A vestibulo-ocular reflex (VOR) eye movement, like nystagmus, can often be induced by a brief period of continuous rotation, for example, 20 seconds of rotation at 30 rpm. VOR can also be studied by using sinusoidally or stochastically varying velocities. Thus, any of a variety of motors can be used to drive the pulley system, including stepper motors or AC motors that are capable of achieving up to or about this rate of angular velocity. A person with skill in the art would be able to determine appropriate devices and systems for achieving adequate, consistent platform rotation and such embodiments are contemplated to be within the scope of the subject invention.

In addition, certain embodiments of the rotary disk 10 may require the motor 22 to be either separate from or affixed to the rotary disk 10, usually depending upon the size and/or type of motor utilized. In a preferred embodiment, as discussed above, a 12V DC motor is used to drive a pulley system 18 to rotate the platform with constant angular velocity 12. In this embodiment, the motor can be preferably fixedly attached to the base 14 of the rotary disk 10. In a more preferred embodiment, the motor is affixed to a motor mounting frame 23 that is connected to the base 14, for example as shown in FIG. 1.

The motor 22 can be controlled by any of a variety of techniques from an “on/off” switch located on or around the motor to wired or radio controlled remote switches 17. In a preferred embodiment, the motor 22, and, thus, the rotation of the platform 12, can be controlled by a remote switch having some operable connection with the motor. In a further preferred embodiment, the rate and direction of rotation can also be controlled by a remote switch having some operable connection with the motor. Alternatively, the rate and direction at which the stepper motor diver turns the platform 12 can be controlled by a computer and/or related software program(s). FIG. 1 shows an example of a wire remote switch 17 for controlling the operation (start, stop, angular velocity, direction, etc.) of a rotary disk 10. However, it should be understood that a person with skill in the art would be able to devise any number of devices for controlling the operation of a rotary disk of the subject invention, including various wireless configurations, and such alternatives are contemplated to be within the scope of the subject invention.

Once a patient is positioned on the platform 12, it can be helpful to ensure that they maintain a safe and consistent position throughout testing. This can permit vestibulo-ocular reflex events to be consistently tested within a patient and among several patients. Thus, an embodiment of the rotary disk 10 of the subject invention further comprises a positioning post 30, for example as shown in FIG. 2.

In a preferred embodiment, a positioning post 30 is fixedly attached to the platform 12 such that it will rotate with the platform. In a more preferred embodiment, the positioning post is attached at or near the platform's axis of rotation. The positioning post 30 can comprise any of a variety of diameters depending upon the type of material utilized and the required strength. In addition, the positioning post can be padded by various methods known in the art, to aid in the comfort and safety of a patient in close proximity. In one embodiment, the positioning post 30 is a generally vertical shaft approximately 1 inch in diameter, which can be padded to approximately 3 inches in diameter. Alternative embodiments can utilize a positioning post having a bent and/or or less vertical form to accommodate different testing positions, or patient comfort, etc. In further embodiments, the positioning post can comprise one or more projections, such as, for example, handles, or other structures for support of a patient or additional equipment, such as a modified infant car seat, etc. In still further embodiments, the positioning post 30 can be removable from the platform 12 such that different positioning posts 30 can be utilized with different patients, testing criteria, etc. In still further embodiments, the position post 30 can be padded, cushioned or otherwise made more comfortable and safe for a patient.

When using the positioning post 30, a patient can sit on the platform 12 with the positioning post, preferably, between the patient's legs. For example, as shown in FIGS. 3 and 4, a patient can be positioned cross-legged on the platform 12 with the positioning post 30 extending between her legs from her lap, such that the patient is facing the positioning post. As will be discussed below, it can be preferable for the height of the positioning post to be adjustable to accommodate patients of different heights and postures. Therefore, in a preferred embodiment, the height of the positioning post 30 is adjustable.

A downward tilt of the head during rotation of the body selectively stimulates the horizontal semicircular canals of the vestibular system eliciting a vestibulo-ocular reflex, usually in the form of horizontal nystagmus. Nystagmus that occurs while rotating is referred to as “per rotary” or “per rotatory” nystagmus and is characterized by repetitive slow eye movement in one direction followed by rapid eye movement in the opposite direction. And, nystagmus that occurs after rotation ceases is referred to as “post rotary” or “post rotatory” nystagmus and is characterized by similar eye movements, but in the opposite direction as the “per rotary” or “per rotatory” direction.

To ensure that a patient is positioned and can maintain the correct head angle, as discussed above, a preferred embodiment of the subject invention can utilize a headrest 30, for example, as shown in FIGS. 2 and 3. The headrest 30 can be angled such that a patient will be facing downward when positioned against the positioning post 30 and head within the headrest 35, for example a shown in FIG. 4. In one embodiment, the headrest is angled from vertical so as to position a patient's head at a downward forward-facing angle between about 20° to about 40° below the horizontal. In a more preferred embodiment, the headrest is angled from vertical so as to position a patient's head at a downward, forward-facing angle between about 25° to about 35°. In a most preferred embodiment, the headrest 30 is angled from vertical so as to position a patient's head at a forward-facing angle of approximately 30°. The headrest can further comprise a head strap 36 to assist in securing a patient's head against the headrest. The headrest can be padded or cushioned for patient comfort and safety. In a preferred embodiment, the headrest 30 comprises a forehead support 37 and a chin support 39. And, in a further preferred embodiment, these supports can be adjustable to fit any size face. The open area between the forehead support 37 and a chin support 39, as shown in FIGS. 3 and 5, accommodates the patient's face and permits easy observation of eye movements.

To provide standard measures for use in clinical assessment or for the diagnosis of vestibulo-ocular disorders, it can be helpful not only to observe and record eye movements, but to also measure the durations of per rotary and post rotary nystagmus. Currently, recordings and measurements are made by the use of electrodes or goggles positioned on the face or over the eyes. This can be uncomfortable and disorienting, particularly when combined with positioning of the head in a headrest and rotation. Certain patients, such as autistic children, who may be particularly in need of testing and evaluation, often cannot tolerate such procedures.

Therefore, one embodiment of the subject invention comprises a camera affixed to the rotary disk that can view and record eye movements. In a preferred embodiment, a video camera is utilized for observing and recording eye movements. However, alternative embodiments can utilize camera-based eye movement monitors. Still other embodiments can utilize optical equipment with higher resolution or better automated data reduction.

In an embodiment using a video camera, for example, as shown in FIGS. 3, 4 and 5, the patient is positioned on the rotary disk with their head placed and secured within the headrest 35 so that the lens 42 of a video camera 40 can be focused on one eye of the patient. Alternative embodiments could utilize additional cameras and/or lenses to record movement in both eyes if necessary or required. Current technology for video cameras often provides a display screen 43, for example, as shown in FIG. 5. A display screen 43 can assist in focusing the camera lens on a patient's eye(s). Any of numerous video cameras and products can be utilized with the embodiments of the subject invention. A person with skill in the art would be able to determine the best video camera, or other recording device, for use with the subject invention and such alternatives are contemplated to be within the scope of the subject invention.

The video camera 40, or other like device, can be affixed to the rotary disk by any of a variety of techniques. In a preferred embodiment, the video camera can rotate with the patient and be sufficiently stable to ensure that it remains focused on a patient's eye(s) during rotation. One embodiment comprises a camera support 44 to affix the camera 40 to the positioning post 30 in front of the headrest 35, for example, as shown in FIGS. 2, 3, 4 and 5. In a further embodiment, the camera support 44 is adjustable and/or pivoting for adjusting the position of the camera 40 to allow focusing on the eyes of various patients.

It can be seen from the above description that the stability of a patient when positioned on the rotary disk of the subject invention can ensure safety as well as accuracy when observing and recording eye movements. The positioning post 30 can provide a stable support for a patient to grip and/or lean against and headrest 35, with the forehead support 37 and chin support 39 ensure that the patient's head and face are properly positioned. However, movement during testing may interrupt the recording process. Furthermore, some patients may not be able to support themselves for very long, or at all.

To ensure that all patients positioned on the rotary device of the subject invention are held securely and that each rotation session can result in accurate observation and recording, a safety vest 50 can be utilized with a patient on the rotary disk 10. A safety vest 50, for example, as shown in FIGS. 3, 4, and 5 can be fitted to the patient and one or more straps 52, preferably at least two straps, can be used to position and secure the patient to the positioning post 30, as shown, for example, in FIGS. 3, 4, and 5. In a preferred embodiment, the safety vest 50 is padded or cushioned for the comfort of a patient and to protect the patient from the pressure of the straps and/or the positioning post 30. Other embodiments can utilize padded straps or other types of harnesses known in the art to secure a patient and are considered within the scope of the subject invention.

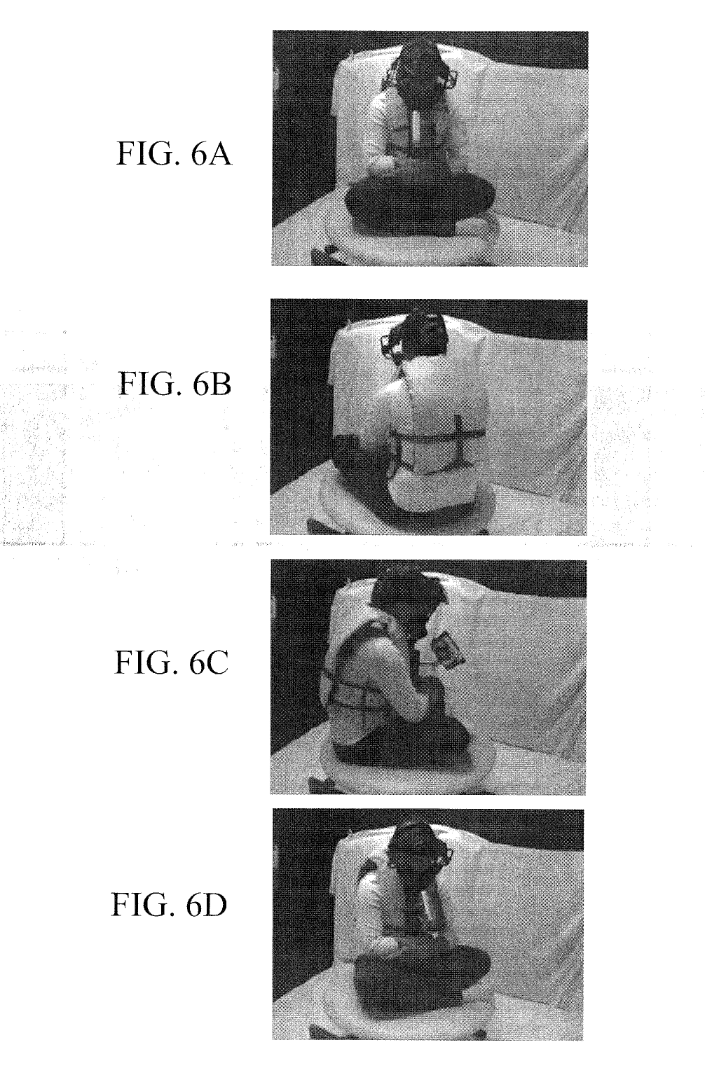

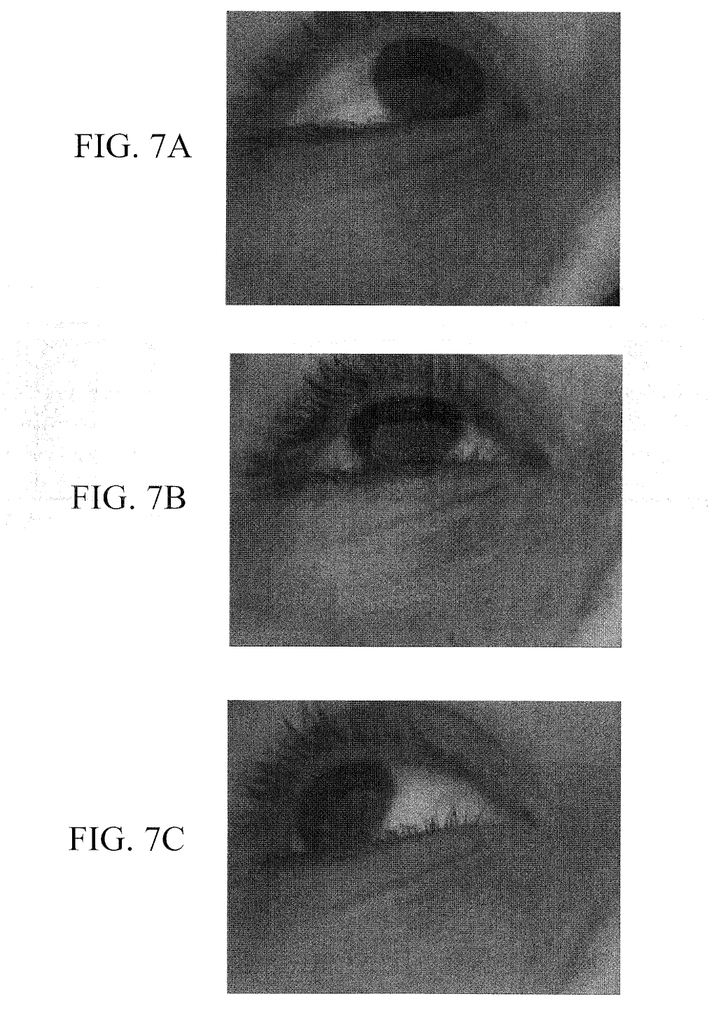

A patient that is properly positioned on a rotary disk 10 of the subject invention and secured with a safety vest 50, can be safely rotated, as shown, for example, in FIGS. 6A-D, at almost any angular velocity. Further, because the patient is stable, with minimal body movement, a video camera 40, or similar device once focused on a patient's eye(s) can views and record an accurate sequence of eye movements, for example, as shown in FIGS. 7A-7B. Because the device is minimally invasive, it is well-tolerated by most patients and can be portable enough to transport.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application

1. A rotary disk device for rotating a patient comprising:
a base;
a platform rotatably connected to the base and having a single axis of rotation;
a positioning post attached to and rotatable with the platform;
a headrest attached to the positioning post; and
a means for viewing and recording patient eye movements, adjustably attached to the rotary disk.

2. The device, according to claim 1, wherein the positioning post is generally vertical and attached to the platform at or near the axis of rotation.

3. The device, according to claim 1, wherein the headrest is tilted such that a patient's head positioned therein is at a downward forward-facing angle.

4. The device, according to claim 1, wherein the headrest is tilted at an angle such that a patient's head positioned therein is at a downward forward-facing angle of approximately 20° to approximately 40° from the horizontal.

5. The device, according to claim 1, wherein the means for viewing and recording is a video camera.

6. The device, according to claim 1, wherein the means for viewing and recording is adjustably attached to the positioning post.

7. The device, according to claim 1, further comprising a safety vest for securing a patient against the positioning post.

8. A rotary disk device for rotating a patient comprising:
a base;
a platform rotatably connected to the base and having a single axis of rotation;
a motor operably connected to the platform that rotates the platform about the axis of rotation;
a positioning post fixedly attached to and rotatable with the platform;
a headrest fixedly attached to the positioning post; and
a means for viewing and recording patient eye movements, adjustably attached to the rotary disk.

9. The device, according to claim 8, wherein the positioning post is attached to the platform at or near the axis of rotation.

10. The device, according to claim 8, wherein the headrest is tilted such that a patient's head positioned therein is at a downward forward-facing angle.

11. The device, according to claim 10, wherein the headrest is tilted at an angle such that a patient's head positioned therein is at a downward forward-facing angle of approximately 20° to approximately 40° from the horizontal.

12. The device, according to claim 8, wherein the means for viewing and recording is a video camera.

13. The device, according to claim 8, wherein the means for viewing and recording is adjustably attached to the positioning post.

14. The device, according to claim 8, further comprising a safety vest for securing a patient against the positioning post.

15. The device, according to claim 8, further comprising a remote device for controlling the motor.

16. A method for eliciting a vestibulo-ocular reflex in a patient utilizing a device comprising:
a base;
a platform rotatably connected to the base and having a single axis of rotation;
a positioning post fixedly attached to and rotatable with the platform;
a headrest fixedly attached to the positioning post;
a safety vest for securing a patient against the positioning post; and
a means for viewing and recording patient eye movements, adjustably attached to the rotary disk,
the method comprising positioning a patient on the platform of the device, securing the patient against the positioning post with the safety vest, positioning the head of the patient within the headrest, rotating the platform, and viewing and recording the patient's eye movements.

17. The method, according to claim 16, wherein the vestibulo-ocular reflex elicited is nystagmus.

18. The method, according to claim 16, wherein the headrest is tilted such that a patient's head positioned therein is at a downward forward-facing angle.

19. The method, according to claim 18, wherein the headrest is tilted at an angle such that a patient's head positioned therein is at a downward forward-facing angle of approximately 20° to approximately 40° from the horizontal.

20. The method, according to claim 18, wherein the means for viewing and recording eye movements is a video camera.

21. The device, according to claim 18, wherein the means for viewing and recording is adjustably attached to the positioning post.
